(19) United States
(12) Patent Application Publication
(10) Pub. No.: US 2026/0324944 A1
(43) Pub. Date: Oct. 22, 2026

(54) MEDICAL DEVICES, SYSTEMS AND RELATED METHODS FOR TRACKING

(71) Applicant: Boston Scientific Scimed, Inc., Maple Grove, MN (US)

(72) Inventors: Kian S. LIM, Shrewsbury, MA (US); Longquan CHEN, Lexington, MA (US); Niraj Prasad RAUNIYAR, Plymouth, MN (US)

(73) Assignee: Boston Scientific Scimed, Inc., Maple Grove, MN (US)

(21) Appl. No.: 19/468,235

(22) Filed: Feb. 3, 2026

Related U.S. Application Data
(60) Provisional application No. 63/753,653, filed on Feb. 4, 2025.

(51) Int. Cl. A61B 1/00; A61B 1/005

(52) U.S. Cl. CPC A61B 1/00006; A61B 1/00057; A61B 1/0052

(57) ABSTRACT

Medical devices, systems, and related methods useful for facilitating navigation through patient anatomy during a medical procedure are described. The medical device may include a handle including at least one actuator, a processor, and a shaft coupled to the handle and defining a channel that extends along a central longitudinal axis. The shaft may include a distal tip, and a coupler proximal to the distal tip. The at least one actuator of the handle may be configured to deflect the distal tip relative to a central longitudinal axis of the shaft. The coupler may include an inertial measurement unit (IMU) sensor, and the processor may be configured to execute a computational model that performs a coordinate transformation using at least one measurement from the IMU sensor. The coordinate transformation may correspond with a calculated position of the distal tip in a base coordinate frame of the handle.

### CROSS-REFERENCE TO RELATED APPLICATION(S)

This application claims the benefit of priority to U.S. Provisional Application No. 63/753,653, filed on Feb. 4, 2025, which is incorporated by reference herein in its entirety.

### TECHNICAL FIELD

Aspects of the present disclosure relate generally to medical devices such as ureteroscope devices and related systems and methods. More specifically, the present disclosure relates to medical systems and devices with sensors to facilitate mapping, navigation, and/or tracking of the distal tip during medical procedures.

### BACKGROUND

During a medical procedure, medical professionals often use medical devices to deflect a distal tip during the medical procedure. Medical professionals will also often use fluoroscopy or similar techniques for tracking a position of the distal tip. However, fluoroscopy risks exposing patients to radiation. Sensors can provide information but space in medical devices is limited, particularly for medical devices used to treat narrow anatomy such as the urological system. It would be useful to have enhanced tracking of the medical device during a medical procedure without exposing the patient to radiation or increasing the size of the medical device.

### SUMMARY

This disclosure relates to, among other things, medical devices and systems for insertion into body lumens and/or other body passageways during medical procedures, and related methods for tracking these devices and systems. Each aspect disclosed herein may include one or more features described in connection with any other disclosed aspect.

According to some aspects, the disclosure provides a medical device that includes a handle including at least one actuator, a processor, and a shaft coupled to the handle and defining a channel that extends along a central longitudinal axis. The shaft includes a distal tip and a coupler proximal to the distal tip. The at least one actuator of the handle may be configured to deflect the distal tip relative to a central longitudinal axis of the shaft. The coupler may include an inertial measurement unit (IMU) sensor, and the processor may be configured to execute a computational model that performs a coordinate transformation using at least one measurement from the IMU sensor. The coordinate transformation may correspond with a calculated position of the distal tip in a base coordinate frame of the handle.

According to some aspects, the handle may include an encoder coupled to the at least one actuator. The encoder may be a rotary encoder or a magnetic encoder, for example. The processor may be configured to execute the computational model using a measurement from the encoder. Further, for example, the processor may be configured to execute a calibration program that calculates a relative position of the coupler in a base coordinate frame and a relative position of the distal tip in the base coordinate frame. The at least one actuator may be a rotary actuator coupled to an encoder that is electrically coupled to the processor, and the processor may be configured to execute the computational model in response to signals received from the encoder. The processor may be configured to generate the computational model based on measurements received from the encoder and the IMU sensor. The coupler may be between a flexible section and an articulation section of the shaft. The coupler may include an electrical port that is electrically coupled to the IMU sensor. A distance between the coupler and the distal tip may be at least 30 mm. The IMU sensor may be coupled to an outer surface and/or an inner surface of the coupler. In some examples, the IMU sensor is received in a cavity of the coupler. Optionally, at least one surface of the IMU sensor may be covered with an epoxy resin.

The disclosure also includes a medical system comprising a medical device and a processor, the medical device comprising a handle that includes an actuator and an encoder coupled to the actuator, and a shaft coupled to the handle and defining a channel that extends along a central longitudinal axis. The shaft includes a distal tip and a coupler proximal to the distal tip, the coupler includes an inertial measurement unit (IMU) sensor, and the at least one actuator of the handle is configured to deflect the distal tip relative to a central longitudinal axis of the shaft. The processor may be configured to execute a computational model using at least one measurement from the IMU sensor. The computational model may perform a coordinate transformation that corresponds with a calculated position of the distal tip in a base coordinate frame of the handle. According to some aspects, the processor may be configured to execute the computational model using the at least one measurement from the IMU sensor and a measurement from the encoder. The coupler may be between a flexible section and an articulation section of the shaft.

The disclosure also provides a method for tracking a distal tip of a medical device. The method may comprise calibrating the medical device, wherein the medical device comprises a handle that includes at least one actuator and an encoder coupled to the at least one actuator, a processor; and a shaft coupled to the handle that defines a channel that extends along a central longitudinal axis of the shaft, wherein the shaft includes the distal tip and a coupler proximal to the distal tip, and wherein the coupler includes an inertial measurement unit (IMU) sensor. The method may include operating the at least one actuator of the handle to deflect the distal tip relative to the central longitudinal axis. The method may include executing, by the processor, a computational model that performs a coordinate transformation using a measurement from the IMU sensor, wherein the coordinate transformation corresponds with a calculated position of the distal tip in a base coordinate frame of the handle. Executing the computational model may include using a measurement from the encoder which corresponds with degree of actuating the at least one actuator.

### BRIEF DESCRIPTION OF FIGURES

The accompanying drawings, which are incorporated in and constitute a part of this application, illustrate aspects of this disclosure and together with the description, serve to explain the principles of this disclosure. The drawings show exemplary medical systems useful for mapping, tracking, and navigation during a medical procedure.

FIG. 1 depicts an exemplary medical device that includes a coupler with a sensor, according to some aspects of the present disclosure.

FIG. 2 depicts the medical device of FIG. 1 used for a medical procedure in a kidney, according to some aspects of the present disclosure.

FIGS. 3 and 4 depict the coupler of the medical device of FIG. 1, according to some aspects of the present disclosure.

FIGS. 5 and 6 depict flow charts of steps for exemplary methods to perform a medical procedure according to some aspects of the present disclosure.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. The terms “proximal” and “distal” are used herein to refer to the relative positions of the components of exemplary medical devices. As used herein, “proximal” refers to a position relatively closer to the exterior of the body or closer to an operator using the medical. In contrast, “distal” refers to a position relatively further away from the operator using the medical device, or closer to the interior of the body.

Aspects of the present disclosure are described herein with reference to exemplary medical devices, systems, and methods that may be useful to facilitate tracking of a distal tip of the medical device during medical procedures. For example, the medical device may include a sensor proximal to the distal tip capable of determining the position of the distal tip during a medical procedure. In some examples, the medical device includes a handle having at least one actuator, and a shaft defining a channel extending distally from the handle to a distal section having a distal tip and a coupler proximal to the distal tip. The distal section may include one or more lighting elements or imaging devices. The distal end of the shaft may be coupled to a cap, for example, including lighting elements or imaging devices and one or more lumens or distal openings (e.g., in communication with a channel of the shaft). The lighting element(s) may include any element that may deliver light to a procedure site, such as light emitting diode(s), optical fibers, or other types of light sources. The imaging device(s) may include a camera and/or other optical device(s) for transmitting an image from a body lumen to a user (e.g., optical fiber, lens, image sensor, etc.). The shaft may include a flexible section and an articulation section distal of flexible section, e.g., the flexible section and the articulation section being connected by a coupler.

The medical device also may include at least one position sensor, e.g., inertial measurement unit (IMU) sensor (e.g., a blend of a gyroscope, magnetometer, and accelerometer), housed within or otherwise located at or coupled to the coupler. In some examples, actuating the at least one actuator of the handle may articulate or deflect the distal tip of the shaft, and the at least one IMU sensor may measure and/or record data associated with motion of the distal tip. The medical device may include, or be electrical and/or wireless communication with, one or more processors configured to execute, and/or to create, at least one computational model using IMU sensor measurements to perform coordinate transformations corresponding with a calculated position of the distal tip. The calculated position may include a calculated coordinate position of the distal tip in a base coordinate frame of the medical device.

References to a particular type of medical procedure, such as a urology procedure; a particular type of medical device, such as a ureteroscope; and/or a particular organ, such as a kidney, are provided for convenience and illustrative purposes, and not intended to limit this disclosure. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration sheaths, catheters, or any other suitable delivery device may be used in connection with the features described herein. Accordingly, analogous medical devices or systems may utilize one or more aspects of exemplary medical devices, systems, and methods described herein.

Reference is now made in detail to examples to help illustrate aspects of the present disclosure through the accompanying drawings. Wherever possible, the same reference numbers will be used throughout the drawings to refer to the same or like parts.

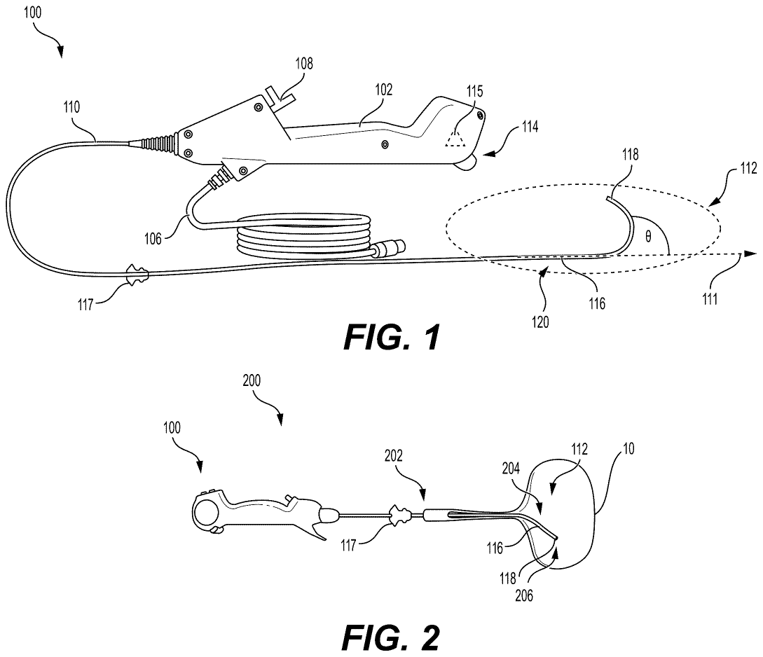

FIG. 1 illustrates an exemplary medical device 100 according to some aspects of this disclosure, medical device 100 including a handle 102 and a shaft 110. The medical device 100 may be configured to allow a user (e.g., medical professional) to deliver medical instruments to access internal areas of a subject's body, e.g., to perform medical diagnoses and/or treatments on the subject. The handle 102 may be coupled to or integral with the shaft 110, such that the handle 102 is in communication with a channel of the shaft 110. Handle 102 may include one or more ports in communication with the channel (and/or other channel(s)) of the shaft 110. For example, the handle 102 may include a port 108 in communication with the channel defined by the shaft 110. Medical instruments such as forceps, graspers, baskets, snares, lasers, and/or other devices may be inserted into the port 108 and advanced through the channel to exit through a distal opening of the shaft 110, e.g., into the subject's body to perform a medical procedure. Additionally or alternatively, the port 108 may be used to provide fluid and/or suction to the channel of the shaft 110 during a medical procedure.

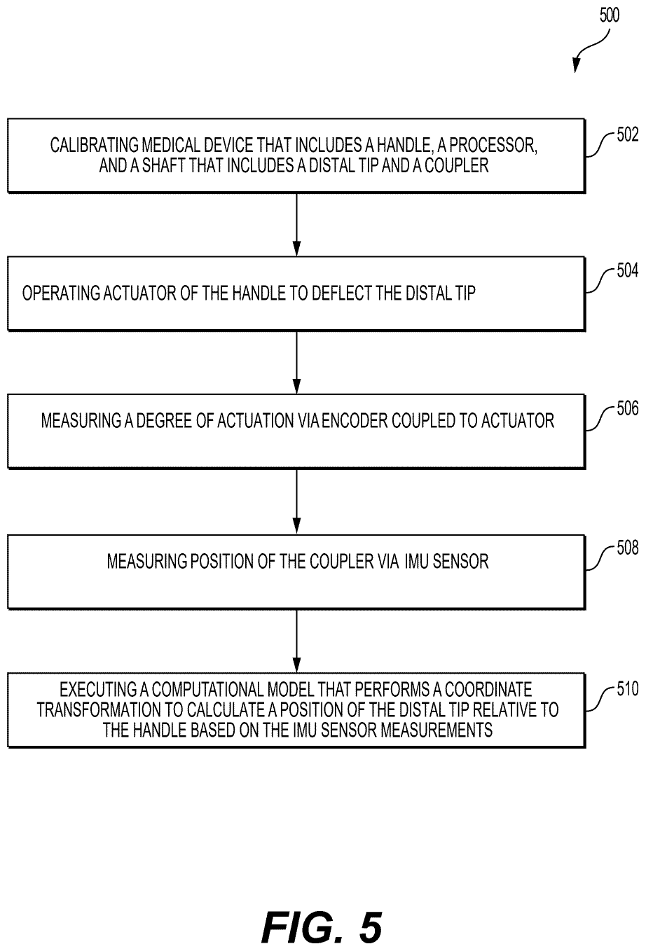

The shaft 110 may extend distally from the handle 102, e.g., along a central longitudinal axis 111 to a distal portion 112 of the medical device 100. The distal portion 112 may have a distal tip 118, e.g., at a distalmost end of the medical device 100. The handle 102 includes at least one actuator 114 coupled to the distal portion 112, such that actuating the actuator 114 may cause the distal tip 118 to deflect, e.g., in one or more directions relative to the central longitudinal axis 111. The shaft 110 also includes a coupler 116 between a flexible section (e.g., a laser-cut tube or other flexible configuration) proximal to the coupler 116 and an articulation section (e.g., including a plurality of articulation segments or joints) distal to the coupler 116, e.g., between the coupler 116 and the distal tip 118. For example, the actuator 114 may rotate, translate, or otherwise move relative to a body of the handle 102 to control one or more pull wires 126 (FIGS. 3 and 4) extending through the shaft (e.g., through the flexible section, the coupler 116, and the articulation section). The handle 102 may also include one or more encoders 115 movably coupled to the actuator 114, e.g., to measure a degree of rotating, translating, or otherwise actuating the actuator 114, e.g., corresponding to deflection degree of the distal tip 118 relative to central longitudinal axis 111.

The coupler 116 includes an IMU sensor 120 positioned proximal of distal tip 118. For example, the coupler 116 and IMU sensor 120 may be separated from the distal tip 118 by an articulation section capable of deflecting the shaft 110 via the actuator 114 of the handle 102. As discussed herein, the user may be able to track and/or map a location of the distal tip 118, e.g., relative to anatomy of a patient during a medical procedure, based on sensor measurements, such as measurements received from one or more sensors of the coupler (from the IMU sensor 120 located at the coupler 116) and/or the handle 102 (e.g., encoder 115 coupled to the actuator 114).

The handle 102 may also include or be coupled to an umbilicus cord 106 for power transmission, e.g., via an external power source, and/or for providing data transmission to or from medical device 100, e.g., such as electrical signals corresponding to measurements transmitted from the encoder 115 and/or IMU sensor 120 and/or auxiliary instruments.

In some aspects, the encoder 115 may be a rotary encoder configured to measure angular position, angular velocity, and/or angular momentum of the actuator 114, e.g., in response to rotating the actuator 114 a degree of rotation that corresponds to degree of deflection of the distal tip 118. In other aspects, the encoder 115 may be a magnetic encoder configured to measure magnetic field changes or disturbances, e.g., in response to actuating the actuator 114 a degree of actuation corresponding to degree of deflection of the distal tip 118. In some examples, the medical device 100 includes two or more encoders, such as one magnetic encoder and rotary encoder each coupled to one or more actuators of the handle 102, such as the actuator 114.

The medical device 100 may also include a linear encoder 117, e.g., coupled to the shaft 110 and configured to measure insertion and retraction of the shaft 110 during medical procedures, e.g., insertion site of the subject's body. For example, the linear encoder 117 may be coupled to a portion of the shaft 110 proximate a location where the shaft 110 enters a subject's body.

The IMU sensor 120 may be capable of measuring acceleration, angular velocity, and/or orientation in three-dimensional space. For example, the IMU sensor 120 may be capable of measuring motion, rotation, and/or other movement of the coupler 116, such as during navigation of the shaft 110 during medical procedures. The IMU sensor 120 may be configured to transmit measurements to one or more processors of or in communication with the medical device 100, for example to execute a computational model to calculate the coordinate position of the distal tip 118 based on the positional measurements associated with coupler 116. The computational model may perform coordinate transformations that include a transformation matrix useful for calculating the distal tip 118 position in the base coordinate frame. In some aspects, measurements from the IMU sensor 120 may be continuously recorded or monitored, e.g., during operation of the medical device 100. As such, the IMU sensor 120 may continuously stream or otherwise provide data associated with multiple axes of motion detection, angular rate, orientation, and/or specific force experienced by the coupler 116 during medical procedures.

FIGS. 3 and 4 depict various views of the distal portion 112 of the shaft 110 including the coupler 116 positioned proximal to the distal tip 118. As shown, the shaft 110 may define a channel 122 extending longitudinally therethrough, e.g., approximately parallel to central longitudinal axis 111, e.g., to provide passage for tools therethrough. For example, the channel 122 may be extend through a length of the shaft 110 to a distal opening at or near a distal-most end of the distal tip 118, such that medical tools can be inserted and extended distally through the channel 122 and through the distal opening. In some examples, the shaft 110 also includes a cable 124, e.g., an electrical cable for transmitting signals from one or more components of the distal portion 112, e.g., a camera or other optical device, lighting element, IMU sensor, etc. As mentioned above, the shaft 110 may also include one or more pull wires 126, for example, which may be coupled to the actuator 114 of the handle 102. The pull wire(s) 126 may extend through the flexible section and articulation section of the shaft 110 and through the coupler 116, e.g., to controlling movement of the distal tip 118 via operation of the actuator 114. As shown, the cable 124 and pull wire(s) 126 may extend adjacent to, e.g., approximately parallel to, the channel 122.

In some examples, the IMU sensor 120 may be coupled to an inner surface or outer surface of the coupler 116. In some examples, the coupler 116 may define a cavity 132 shaped and sized to receive the IMU sensor 120. In such cases of being disposed within the cavity 132 and/or being coupled to a surface of the coupler 116, the IMU sensor 120 may be over-molded into coupler 116. For example, the IMU sensor 120 may be partially or completed covered with a molding material such as epoxy resin. The IMU sensor 120 may include two or more electrically coupled components, such as an IMU die, electrical wiring, and/or a casing. The casing may include an epoxy resin having an IMU die embedded therein, such that resultant shape of molded resin complements that of the shaft 110 and/or the distal tip 118.

In some aspects, the IMU sensor 120 may be a fixed position from the distal tip 118, e.g., separated by an articulation section of the shaft 110, e.g., a distance between the coupler 116 and the distal tip 118. For example, the IMU sensor 120 may be at least 30 millimeters (mm) away from the distal tip 118 relative to the central longitudinal axis 111, e.g., from about 30 mm to about 70 mm, from about 40 mm to about 60 mm, or about 50 mm. In at least one example, the IMU sensor 120 is about 50 mm away from the distal tip 118 relative to the central longitudinal axis 111. In some aspects, the IMU sensor 120 may have dimensions that are approximately 2.0 mm×2.0 mm×1.0 mm (length×width×height).

Coupler 116 may include an electrical port electrically coupled to the IMU sensor 120 that facilitates communication between the IMU sensor 120 and other components of the medical device 100, such as the handle 102. In some cases, the coupler 116 may define one or more channels to accommodate electrical components such as wiring to electrically couple the IMU sensor 120, the electrical port, and/or the handle 102.

Encoder 115, linear encoder 117, and/or IMU sensor 120 may be electrically or wirelessly coupled to one or more processors, which may provide real-time data processing capability for the medical device 100. The processor(s) may be housed within the medical device 100 (e.g., within the handle 102) and/or may be external to the medical device 100 and in wired or wireless communication with encoder 115, linear encoder 117, and/or IMU sensor 120. In some cases, the processor(s) may be configured to execute a computational model using measurements from encoder 115 and/or IMU sensor 120. Such measurements may be useful as input to the computational model for performing one or more matrix transformations, e.g., for calculating a coordinate position of the distal tip 118 in the base coordinate frame. For example, during a medical procedure, the user may operate the actuator 114, which in turn may deflect the distal tip 118 relative to central longitudinal axis 111 and cause encoder 115 to measure degree of movement, e.g., rotation, of the actuator 114. The computational model may use the measurement of encoder 115 as input, e.g., corresponding to degree of deflection of distal tip 118 in response to actuating the actuator 114 of handle 102, to calculate a coordinate position of distal tip 118 in the base coordinate frame. In some aspects, the computational model may associate a degree of actuation (e.g., of actuator 114 measured via encoder 115) with degree of deflection (e.g., of the distal tip 118 relative to central longitudinal axis 111). This association may be derived, for example, from historical signal data that includes coordinate position data for the coupler 116 (e.g., measured via IMU sensor 120) as the distal tip 118 bends or otherwise deflects in response to operation of actuator 114. Computational models therefore may use a position of the actuator 114 as input, which may be derived from data measured by encoder 115, and in turn calculate the position of the distal tip 118 using coordinate transformations.

In some examples, the computational model may use measurements from encoder 115 and/or IMU sensor 120 as input to calculate the position of distal tip 118. For instance, the computational model may calculate the position of distal tip 118 using measurements of encoder 115 corresponding to operation of the actuator 114 and measurements of IMU sensor 120 corresponding to a position of the coupler 116 after operation of the actuator 114. These measurements may be used to perform coordinate transformations between various coordinate frames of the medical device 100, such as a base frame of the handle 102, a coupler frame of the coupler 116, and a tip frame of the distal tip 118. In some aspects, these calculations may be performed in real-time, such that real-time processing of sensor measurements allows for real-time calculating of the position of distal tip 118, e.g., by having the model perform coordinate transformations using measurements. Combining the measurements of IMU sensor 120 with other sensor data, such as measurements of encoder 115 in response to operation of the actuator 114 may allow the model to perform coordinate transformations, e.g., for mapping and tracking the location of distal tip 118 and thereby enhance user ability to navigate the subject's body during medical procedures using the medical device 100.

In aspects, the measurements of encoder 115 may provide information about movements controlled by the handle 102, while data from the IMU sensor 120 may account for other movements of medical device 100. In some aspects, the medical device 100 may be configured to execute a calibration process, for example using measurements from the encoder 115 and/or linear encoder 117 to calibrate the IMU sensor 120. Calibration may include correlating known positions of actuator 114 measured via the encoder 115 with measurements of IMU sensor 120 that correspond to the position of coupler 116. In some cases, calibration may also include correlating linear motion (e.g., advancement or retraction) of shaft 110 via the linear encoder 117 with measurements of encoder 115 and/or IMU sensor 120 that correspond to the position of coupler 116. For example, calibrating the medical device 100 may be performed when the shaft 110 is in a neutral, undeflected position, e.g., substantially parallel to the central longitudinal axis 111.

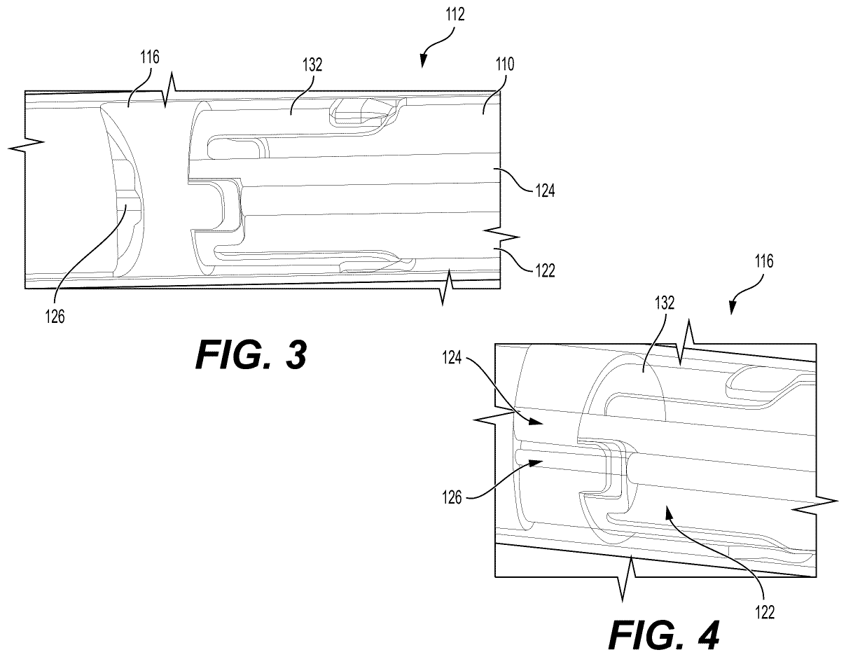

FIG. 2 illustrates medical device 100 used in an exemplary medical procedure, e.g., inserted into a kidney 10 of a subject's body. As shown, the medical device 100 may be part of a medical system 200 (e.g., further comprising one or more processors, displays, etc.) defining a plurality of coordinate frames during the medical procedure. In this example, a base coordinate frame is defined as the entry point of the medical device 100 into patient anatomy, e.g., having a base origin 202 at an insertion site during the medical procedure, e.g., the base origin 202 at or distal the location of linear encoder 117 along the shaft 110. A coupler coordinate frame is defined at the coupler 116 of the shaft 110, e.g., having a coupler origin 204 at the location of the IMU sensor 120 in the neutral, undeflected state of the shaft 110. A tip coordinate frame is defined at the distal tip 118, e.g., having a tip origin 206 at the location of the distal tip 118 in the neutral, undeflected state of the shaft 110. By relating the position of the distal tip 118 to a fixed reference point of the IMU sensor 120 at the coupler 116, the medical device 100 may provide more accurate tracking of movement of the distal tip 118 within the subject's body during medical procedures.

One or more processors of the medical device 100 and/or system 200 may be in communication with the encoder 115 and/or IMU sensor 120, the processor(s) configured to execute computational models using measurements from the encoder 115 and/or IMU sensor 120. For example, the computational models may be used to calculate the position of the distal tip 118 relative to the base origin 202, e.g., the coordinate position of the distal tip 118 in the base coordinate frame.

In some aspects, the processor(s) may be configured to execute a visual-inertial SLAM algorithm, which may utilize data from IMU sensor 120 and images captured at the distal tip 118, e.g., from a camera or other optical sensor of the distal tip 118. In some examples, SLAM may utilize one or more other data sources, e.g., measurement data from encoder 115 and/or linear encoder 117. Combining data sources, e.g., images and data from the IMU sensor 120, may be useful to enhance tracking and mapping capability of the subject's body and facilitate navigation during medical procedures. SLAM may provide real-time 3D mapping and localization of the medical device 100 during medical procedures, e.g., of the distal tip 118 within the subject's body.

In some aspects, the processor(s) may be configured to execute a calibration program which, for example, may calibrate the shaft 110 while the shaft 110 is undeflected extending substantially along the central longitudinal axis 111. During calibration, the processor(s) may calculate a position of the coupler 116 and IMU sensor 120 relative to the base coordinate frame, e.g., calibrating the coupler origin 204 relative to the base origin 202 while the shaft 110 is undeflected. Additionally, the calibration process may determine a relative position of the distal tip 118 relative to the coupler coordinate frame and/or the base coordinate frame. For example, the process may include calibrating the tip origin 206 relative to the coupler origin 204 and/or the base origin 202. Calibration may include calculating coordinate positions for each of the coupler 116 and the distal tip 118 based on measurements of the IMU sensor 120 and known positions of medical device 100 components when the shaft 110 is in a neutral position.

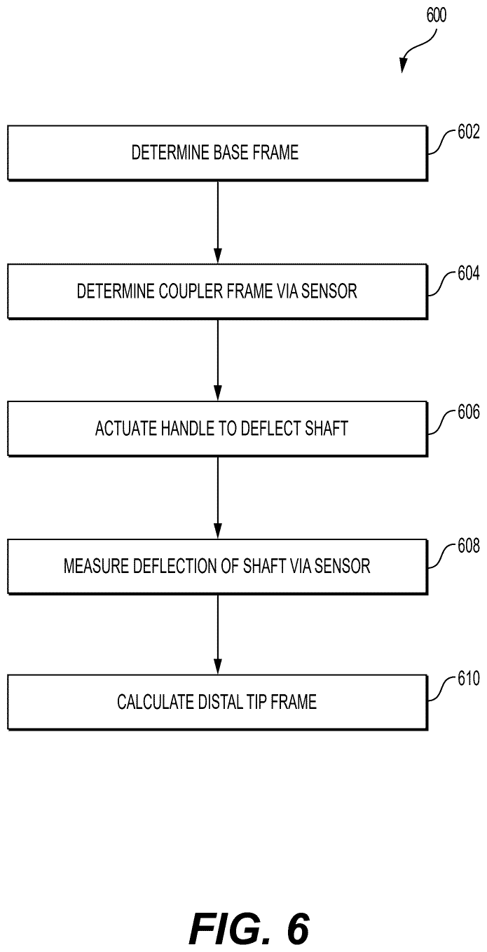

FIG. 5 illustrates a flow chart of an exemplary method 500 for performing a medical procedure, according to aspects of this disclosure. The method 500 may be implemented using a medical device that includes an IMU sensor proximal to the distal tip of the medical device, such as the medical device 100 shown in FIGS. 1-4.

Step 502 of the method 500 comprises calibrating a medical device that includes a handle, a processor, and a shaft with a distal tip and a coupler that includes an IMU sensor. Calibrating the medical device may comprise calculating an undeflected position of the distal tip relative to a base coordinate frame of the handle. For example, when calibrating the medical device 100, the shaft 110 extends along the central longitudinal axis 111 in an undeflected state, and the processor may calculate the coordinate position of the distal tip 118 in the base coordinate frame. In some implementations, step 502 may also include calculating the relative position for the coupler 116 (including the relative position of the IMU sensor 120) and distal tip 118 in the base coordinate frame, for example transformed from either the coupler or tip coordinate frames.

Next, step 504 includes actuating at least one actuator of the handle to deflect the shaft and distal tip. For instance, a user may actuate the actuator 114 to deflect the distal tip 118 at the distalmost end of the medical device 100.

In step 506, the method 500 includes measuring the degree of actuation via an encoder coupled to the actuator. For example, the encoder 115 coupled to actuator 114 may measure a degree of actuation in response to operating the actuator 114 in step 504.

Step 508 comprises measuring the relative position of the coupler and IMU sensor using data measured by the IMU sensor. In the case of medical device 100, measuring the coordinate position of coupler 116 in the coupler coordinate frame may be based on measurements from the IMU sensor 120, e.g., in response to the deflection of distal tip 118 from step 504.

In step 510, method 500 includes executing a computational model that performs coordinate transformations to calculate the position of the coupler 116 relative to the handle 102 based on IMU sensor measurements. For example, the processor may execute the model to determine the position of the coupler 116 in the three-dimensional base coordinate frame (x, y, z) using the following transformation matrix (Equation 1):

$${T}_{tail}^{base} = [ \begin{matrix}R & Pos \\ 0 & 1\end{matrix} ] = [ \begin{matrix}[ \begin{matrix}{X}_{x} & {Y}_{x} & {Z}_{x} \\ {X}_{y} & {Y}_{y} & {Z}_{y} \\ {X}_{z} & {Y}_{z} & {Z}_{z}\end{matrix} ] & \begin{matrix}X \\ Y \\ Z\end{matrix} \\ \begin{matrix}0 & 0 & 0\end{matrix} & 1\end{matrix} ] \qquad Equation 1$$

To calculate position of the distal tip 118, step 510 may include data from both of the encoder 115 and IMU sensor 120, e.g., as inputs for the computational model to perform coordinate transformations and calculate the position of distal tip 118. For example, the processor may execute the model to calculate coordinate position of distal tip 118 to the coupler coordinate frame after movement of the actuator 114 to deflect the distal tip 118, using the following equations:

$$\hat{z} = \frac{( {\vec{V}}_{1} \times {\vec{V}}_{2} )}{( {\vec{V}}_{1} \times {\vec{V}}_{2} )} \qquad Equation 2$$

$$\hat{x} = \frac{( tip 2 - tip 1 ) \times \hat{z}}{\vert ( tip 2 - tip 1 ) \times \hat{z} \vert} \qquad Equation 3$$

$$\hat{y} = \hat{z} \times \hat{x} \qquad Equation 4$$

$${T}_{tip}^{0} = \begin{matrix}\hat{x} & \hat{y} & \hat{z} & tip 1 \\ 0 & 0 & 0 & 0\end{matrix} \qquad Equation 5$$

$${T}_{tip}^{base} = {T}_{tail}^{base} * {T}_{tip}^{tail} \qquad Equation 6$$

In this example, [symbol] and [symbol] include outputs from a function that computes center and radii of circles, that interpolate some given triples of points in three-dimensional space. This function accepts measurements of encoder 115 and IMU sensor 120, e.g., in response to movement of actuator 114, including input of matrices respectively corresponding to degree of movement of actuator 114 and to degree of movement of coupler 116. For example, the processor may use [symbol] and [symbol] to execute Equation 2, the output of which may be used to execute Equation 3, the output of which may be used to execute Equation 4. The processor may further use outputs from Equations 2-4 as respective inputs to execute Equation 5, which corresponds to the calculated position of distal tip 118 in the coupler coordinate frame. The output of Equation 5 may be used to execute Equation 6, which corresponds to the calculated position of distal tip 118 in the base coordinate frame. Method 500 therefore may provide tracking of medical devices during medical procedures.

FIG. 6 illustrates various steps for performing another exemplary method 600 according to aspects of this disclosure. At step 602, method 600 may include determine a base frame, for example, determining the base origin 202 of the base coordinate frame of the medical device 100 using the calibration process (e.g., step 502 of method 500).

At step 604, method 600 may include determining a coupler frame via sensors of the medical device, for example determining the coupler origin 204 of the coupler frame of the medical device 100 using data measured by IMU sensor 120. At step 606, method 600 may include actuating the handle of the medical device to deflect the shaft. For example, a user may operate the actuator 114 of the handle 102 to deflect the distal tip 118 of the shaft 110 relative to the central longitudinal axis 111. At step 608, method 600 may include measuring deflection of the shaft via sensor data, for example measuring a degree of movement of the actuator 114 via data measured by encoder 115.

At step 610, method 600 may include executing a computational model to perform coordinate transformations that calculate a coordinate position of the distal tip 118 relative to the base origin 202 of the base coordinate frame.

In some examples, the processor may generate a computational model, for example using a historical dataset comprising data measured by the encoder 115 and/or the IMU sensor 120 from previous medical or experimental procedures. Such data may be saved within the processor or otherwise retrieved by the processor from another storage medium. For example, the dataset may include measurements made by IMU sensor 120 corresponding to a first position of the coupler 116 at a first time point, measurements made by encoder 115 corresponding to operation of the actuator 114 at a second time point, and measurements made by IMU sensor 120 corresponding to a second position of the coupler 116 at a third time point.

In this example, the first position of the coupler 116 may represent the shaft 110 in a neutral, undeflected position substantially parallel to the central longitudinal axis 111. Further, the second position of the coupler 116 may represent the shaft 110 after deflection such that the distal tip 118 is deflected relative to the central longitudinal axis 111, e.g., in response to operation of actuator 114. The dataset may have multiple iterations of similar measurement data from multiple sources, which may be increase accuracy of the final computational model. In some examples, the computational model is configured to be executed using one of the measurements of encoder 115 or the IMU sensor 120. For example, the computational model may perform coordinate transformations based on degree of movement of actuator 114, such as calculations that transform coordinates between two or more of the base coordinate frame, coupler coordinate frame, and the tip coordinate frame.

Medical devices, systems, and related methods for performing medical procedures discussed in this disclosure may increase the working volume in the distal tip during medical procedures, e.g., due to use of positioning sensors, such as IMU sensors and/or encoders, proximal of the distal tip. This may allow for additional components to be positioned within the distal tip, and/or different arrangements of components therein, yet retain the ability to accurately track the position of the distal tip of the shaft during medical procedures. Aspects of the present disclosure, for example, may lead to better visualization or reduce procedure time, among other improvements to patient experience or result of minimally invasive procedures.

While principles of this disclosure are described herein with reference to illustrative examples, it should be understood that the disclosure is not limited thereto. Those having ordinary skill in the art and access to the teachings provided herein will recognize additional modifications, embodiments, and substitution of equivalents all fall within the scope of the features described herein. Accordingly, the claimed features are not to be considered as limited by the foregoing description.

We claim:

1. A medical device, comprising:
a handle including at least one actuator;
a processor; and
a shaft coupled to the handle and defining a channel that extends along a central longitudinal axis, wherein the shaft includes a distal tip and a coupler proximal to the distal tip;
wherein the coupler includes an inertial measurement unit (IMU) sensor;
wherein the at least one actuator of the handle is configured to deflect the distal tip relative to a central longitudinal axis of the shaft; and
wherein the processor is configured to execute a computational model that performs a coordinate transformation using at least one measurement from the IMU sensor, wherein the coordinate transformation corresponds with a calculated position of the distal tip in a base coordinate frame of the handle.

2. The medical device of claim 1, wherein the handle includes an encoder coupled to the at least one actuator.

3. The medical device of claim 2, wherein the processor is configured to execute the computational model using a measurement from the encoder.

4. The medical device of claim 2, wherein the encoder is a rotary encoder.

5. The medical device of claim 2, wherein the encoder is a magnetic encoder.

6. The medical device of claim 1, wherein the processor is configured to execute a calibration program that calculates a relative position of the coupler in a base coordinate frame and a relative position of the distal tip in the base coordinate frame.

7. The medical device of claim 6, wherein the at least one actuator is a rotary actuator coupled to an encoder that is electrically coupled to the processor, and wherein the processor is configured to execute the computational model in response to signals received from the encoder.

8. The medical device of claim 7, wherein the processor is configured to generate the computational model based on measurements received from the encoder and the IMU sensor.

9. The medical device of claim 1, wherein the coupler is between a flexible section and an articulation section of the shaft.

10. The medical device of claim 1, wherein the coupler includes an electrical port that is electrically coupled to the IMU sensor.

11. The medical device of claim 1, wherein a distance between the coupler and the distal tip is at least 30 mm.

12. The medical device of claim 1, wherein the IMU sensor is coupled to an outer surface of the coupler.

13. The medical device of claim 1, wherein the IMU sensor is coupled to an inner surface of the coupler.

14. The medical device of claim 1, wherein the IMU sensor is received in a cavity of the coupler.

15. The medical device of claim 1, at least one surface of the IMU sensor is covered with an epoxy resin.

16. A medical system, comprising:
a medical device, comprising:
a handle including an actuator and an encoder coupled to the actuator, and
a shaft coupled to the handle and defining a channel that extends along a central longitudinal axis,
wherein the shaft includes a distal tip and a coupler proximal to the distal tip, wherein the coupler includes an inertial measurement unit (IMU) sensor,
wherein the actuator of the handle is configured to deflect the distal tip relative to a central longitudinal axis of the shaft; and
a processor configured to execute a computational model using at least one measurement from the IMU sensor, wherein the computational model performs a coordinate transformation that corresponds with a calculated position of the distal tip in a base coordinate frame of the handle.

17. The medical system of claim 16, wherein the processor is configured to execute the computational model using the at least one measurement from the IMU sensor and a measurement from the encoder.

18. The medical system of claim 17, wherein the coupler is between a flexible section and an articulation section of the shaft.

19. A method for tracking a distal tip of a medical device, the method comprising:
calibrating the medical device, wherein the medical device comprises:
a handle that includes at least one actuator and an encoder coupled to the at least one actuator,
a processor; and
a shaft coupled to the handle that defines a channel that extends along a central longitudinal axis of the shaft, wherein the shaft includes the distal tip and a coupler proximal to the distal tip, and wherein the coupler includes an inertial measurement unit (IMU) sensor;
operating the at least one actuator of the handle to deflect the distal tip relative to the central longitudinal axis; and
executing, by the processor, a computational model that performs a coordinate transformation using a measurement from the IMU sensor, wherein the coordinate transformation corresponds with a calculated position of the distal tip in a base coordinate frame of the handle.

20. The method of claim 19, wherein executing the computational model includes using a measurement from the encoder which corresponds with degree of actuating the at least one actuator.
